(19) United States
(12) Patent Application Publication
(10) Pub. No.: US 2015/0157620 A1
(43) Pub. Date: Jun. 11, 2015

(54) LOZENGES WITH MULTIPLE RELEASE KINETICS FOR ACTIVE INGREDIENTS

(71) Applicant: PIERRE FABRE MEDICAMENT, Boulogne-Billancourt (FR)

(72) Inventors: Didier Berthoumieu, Villenouvelle (FR); Clément Laur, Lescout (FR); Jean-François Cordoliani, Sainte Foy d'Aigrefeuille (FR)

(73) Assignee: PIERRE FABRE MEDICAMENT, Boulogne-Billancourt (FR)

(21) Appl. No.: 14/418,327

(22) PCT Filed: Jul. 30, 2013
(86) PCT No.: PCT/EP2013/066032

§ 371 Date: Jan. 29, 2015

(30) Foreign Application Priority Data
Jul. 30, 2012 (FR) 1257389

(51) Int. Cl. A61K 31/465; A61K 9/00; B05D 1/02; A61K 9/24

(52) U.S. Cl. CPC A61K 31/465; A61K 9/209; A61K 9/0056; B05D 1/02

(57) ABSTRACT

The present invention relates to a film-coated lozenge intended to dissolve in the oral cavity, comprising a core comprising at least one active ingredient and at least one cooked sugar, and a shell comprising at least one layer comprising at least one active ingredient.

The present invention relates to a film-coated lozenge intended to dissolve in the oral cavity and enabling an active ingredient to be administered orally as well as its use as a drug.

Dosage forms enabling an active ingredient to be administered orally are particularly useful for administering active ingredients likely to be degraded upon ingestion and/or likely to undergo a hepatic first pass effect but that can enter the bloodstream by passing through the oral mucosa.

Nicotine is a highly addictive substance present in cigarettes. The majority of smokers experience difficulties in refraining from or stopping smoking because they soon experience withdrawal feelings. Nicotine supply by a tobacco substitute allows this withdrawal to be treated and thus stopping smoking to be promoted.

These substitutes can be in different dosage forms in particular chewing gums, sublingual tablets, inhalers, suckable tablets or suckable lozenges.

Chewing gums, suckable tablets and suckable lozenges have the advantage, besides nicotine supply, of distracting the attention of the patient stopping smoking by a physical action: chewing or sucking which promotes forgetting the crave for cigarette.

Chewing gums (such as especially described in patent U.S. Pat. No. 3,877,468) have a number of disadvantages, in particular, as regards the absorbed nicotine dose. Indeed, it is difficult to regulate the total amount of nicotine released by a chewing gum as well as its release kinetics, these are strongly influenced by the use made by the patient. Chewing indications (a few mastications and then stopping and holding the gum against the cheek) are rarely respected by the patients. On the contrary, they have a tendency to strongly chew the gum at the beginning of the use when the desire to smoke is strongest. This results in too quick a release of nicotine, too high an amount of nicotine through the digestive tract (where it will be predominantly degraded) and thus a sub-dosage of nicotine in the remaining gum. This too quick nicotine ingestion further generates numerous side effects (nausea, stomach pain, unpleasant taste). Chewing is further unsightly and deleterious to dental prostheses.

Cooked sugar based suckable lozenges, in particular sold under the brand Nicopass® (see in particular WO2002/085334), allow problems related to chewing gums to be avoided in particular by evenly and fully delivering nicotine for about 30 minutes, without the possibility for the user to speed-up dissolving the lozenge.

Lozenges can however be not fully suitable for heavy smokers or strongly dependant smokers. Indeed, in this kind of patients, the desire to smoke is particularly strong within the first minutes and then decreases little by little. Chewing lozenges currently commercially available can be considered as too slow to treat the first desire to smoke in these patients.

Thus, there is currently a real need for an alternative nicotine substitute allowing an even nicotine diffusion for a long enough time for the desire to smoke to have fully disappeared and sufficiently rapidly to make up for the early desire to smoke experienced by heavy smokers or strongly dependent smokers. It is further preferable for a good treatment follow-up that the substitute has a pleasant taste and that it generates no or few side effects.

### FIGURES

FIG. 1: schematic view of a side cross section of a lozenge according to invention comprising a core, a shell consisting of an inner isolating layer, a layer comprising at least one active ingredient and an outer isolating layer as well as an outer layer coating the shell.

FIG. 2: schematic view of a side half cross section of a lozenge according to invention comprising a core, a shell consisting of alternating isolating layers and layers comprising at least one active ingredient as well as an outer layer coating the shell.

FIG. 3: nicotine release kinetics of a lozenge C (square) and D (diamond).

Surprisingly in the present invention, a new dosage form was developed intended to dissolve in the oral cavity enabling an active ingredient, in particular nicotine, to be administered, according to a double kinetics: quick within the very first minutes after being taken by the patient and then sustained until the end of the withdrawal feeling.

The present invention thus relates to a film-coated lozenge intended to dissolve in the oral cavity comprising:
a core comprising at least one active ingredient and at least one cooked sugar,
a shell comprising at least one layer comprising at least one active ingredient.

In one embodiment, the shell further comprises at least one layer comprising a film-forming polymer.

In one embodiment, the film-coated lozenge according to the invention further comprises an outer layer coating the shell.

By “lozenge” according to the invention, it is meant a very specific dosage form, that was in particular the subject of a monograph of the French pharmacopeia Xth edition (July 1987). The lozenges are saccharoids having a solid a hard consistency obtained by a cooking operation of a polyol and intended to slowly dissolve in the oral cavity. The lozenges according to the invention are coated or film-coated lozenges which preferentially have a round flat shape or a ball shape. The term lozenge according to the invention both corresponds to the English terms “lozenge” or “hard candy”. By film-coated, it is meant that the lozenge according to the invention is coated with a shell and possibly an outer layer. The shell and the possible outer layer will be preferentially thin that is the thickness thereof is lower than 1 mm, preferentially lower than 0.1 mm, preferentially between 1 μm and 1 mm, further preferentially between 10 μm and 0.1 mm.

By “intended to dissolve in the oral cavity”, it is meant that the lozenge according to the invention is capable of dissolving in the mouth in contact with saliva. The lozenge according to the invention enables the entire active ingredient it contains to be released in the oral cavity. The lozenge according to the invention will thus have only an oral dissolution and will not be intended to be swallowed. Preferentially, the core of the lozenge according to the invention will be sufficiently resistant so that it will be impossible or difficult for the patient to crunch this lozenge and thus to swallow pieces thereof (the strength of the lozenge could be a function of its hardness, for example the lozenge according to the invention could have a hardness between 300 and 700N, for example between 400 and 600N as measured by a Schleuniger type durometer. The patient is then obliged to suck the lozenge until its disappearance which ensures the constant regular nicotine supply.

In one embodiment, the core of the lozenge according to the invention will have a total dissolution time between 15 and 35 minutes, preferentially between 15 and 30 minutes, further preferentially between 25 and 30 minutes, for example 30 minutes.

In one embodiment, the lozenge according to the invention will have a total dissolution time between 15 and 40 minutes, preferentially between 15 and 30 minutes, further preferentially between 25 and 30 minutes, for example 30 minutes.

The core of the lozenge according to the invention comprises at least one cooked sugar. By cooked sugar, it is meant a sugar obtained through cooking a polyol or simple sugar.

The polyol will be preferentially selected from the group comprising or consisting of saccharose, fructose, lactose, sorbitol, mannitol, lactitol, glucose, isomalt, polydextrose and maldodextrins or mixtures thereof, preferentially isomalt. The polyol according to the invention will preferably represent 80 to 97 weight % of the lozenge, preferentially between 85 and 95%, for example around 90%.

Techniques of obtaining a cooked sugar according to the invention are well known to those skilled in the art and are in particular described in “sugar confectionery and manufacture” (E.B. Jackson (Springer, 1995)). WO2002/085334 also discloses the manufacture of this formulation type, thus able to be applied to the present invention. The conditions selected to obtain the cooked sugar could depend on several parameters in particular the temperature, pressure, concentration of sugar and of water. For example, the sugar according to the invention could be cooked at a temperature between 140 and 200° C., for example 150 and 180° C. at atmospheric pressure. The sugar after cooking is in a vitreous state.

In order to increase the strength thereof, the core of the lozenge according to the invention could contain a matrix agent. Preferred matrix agents are preferentially selected from the group comprising or consisting of non-cellulosic polysaccharides, cellulosic derivatives, acrylic acid polymers, fats, polyvinylpyrrolidone or mixture thereof, preferentially polyvinylpyrrolidone. Preferentially, the matrix agent will be selected from those described in patent application WO2002/085334. In particular, the matrix agent could be typically selected from the group comprising or consisting of non-cellulosic polysaccharides, cellulosic derivatives (in particular hydroxypropylcellulose, hydroxypropylmethylcellulose, hydroxyethylcellulose, ethylcellulose), acrylic acid polymers, fats, polyvinylpyrrolidone, gums or mixtures thereof. In one embodiment, the matrix agent represents between 1 to 10 weight % of the lozenge, typically 1 to 5%.

The core of the lozenge according to the invention could further comprise at least one auxiliary substance selected in particular from sweeteners, buffering agents, antioxidants, colorants or aromas. The core of the lozenge is intended to be hard in order to be able to be sucked. The core will thus comprise preferentially few or no materials such as gum, pectins or other texturing agents making the lozenge insufficiently hard of the “chewing gum” type.

In embodiments of the invention, the core of the lozenge will be such as defined in application WO2002/085334 (described as a “lozenge”) and in particular as described in examples 1 to 9 of application WO2002/085334, or in the examples of the present application.

By “active ingredient” according to the invention, it is meant any active ingredient that can be administered orally, intramucosally, sublingually or perlingually with a systemic or local aim that is buccal or pharyngeal. As an active ingredient according to the invention, there can be mentioned in particular anti-inflammatory agents such as ibuprofen, flurbiprofen, enoxolone and ketoprofen, anesthetics such as lidocaine, tetracaine and ambroxol, antiseptics such as CHX, biclotymol and benzydamide, antitussives in particular central ones such as dextromethorphan and pholcodine as well as miconazole, chlorhexidine, arginine, iron, vitamins, nicotine, bases, salts or complexes thereof and combinations thereof.

Preferentially, the active ingredient according to the invention is nicotine, as a salt, base or complex. As a nicotine salt, there can be mentioned in particular nicotine ditartrate (also called bitartrate), in particular nicotine ditartrate dihydrate. According to one embodiment, the nicotine is selected from the group consisting of nicotine ditartrate and nicotine resinate. Preferentially, the nicotine comprised in the core is nicotine resinate and the nicotine comprised in the layer of the shell comprising the active ingredient is nicotine bitartrate.

By “shell” according to the invention, it is meant a set of one or more identical or different layers, at least one of which is a layer comprising at least one active ingredient.

In an alternative, the shell consists of a single layer comprising at least one active ingredient, the terms “shell” and “layer comprising at least one active ingredient” are then equivalent.

In another alternative, the shell comprises several layers comprising at least one active ingredient (for example 2, 3, 4 or 5). In one embodiment, each of these layers comprises a single active ingredient, that can be identical or different from that present in the other layer(s) comprising an active ingredient.

By “inner layer” according to the invention, it is meant a layer sequentially closer to the core with respect to an “outer layer”.

By “outer layer” according to the invention, it is meant a layer sequentially further from the core with respect to an “inner layer”.

By “sequentially”, it is meant the passage from one layer to another, in the manner of a sequence of layers.

In another alternative, the shell according to the invention further comprises at least one isolating layer. By “at least one isolating layer” for the purposes of the present invention, it is meant one, two, three or more isolating layers. Preferentially, the lozenge according to the invention will comprise at least one isolating layer adjacent to the core, that is directly in contact with the core and/or at least one isolating layer adjacent to the outer layer, that is directly in contact with the outer layer.

In a preferred embodiment, the isolating layers do not comprise an active ingredient.

When the shell according to the invention comprises more than one isolating layer, “inner isolating layer” will designate the isolating layer(s) sequentially closest to the core with respect to the “outer isolating layers” and “outer isolating layer” will designate the isolating layer(s) sequentially furthest from the core with respect to the “outer isolating layer(s)”. According to one embodiment, the shell comprises at least one inner isolating layer and/or one outer isolating layer.

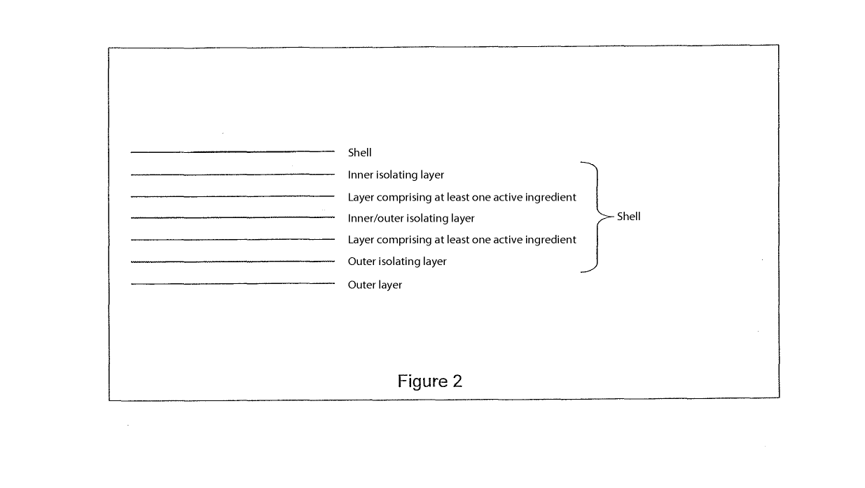

In one embodiment, the shell comprises alternating isolating layers and layers comprising at least one active ingredient. An exemplary embodiment is illustrated in FIG. 2. It can be noted that in the embodiment illustrated in FIG. 2, the isolating layer in the shell located between two layers comprising at least one active ingredient is both an inner and outer isolating layer.

In one embodiment, the shell consists of an inner isolating layer, a layer comprising at least one active ingredient and an outer isolating layer.

In another embodiment, the shell comprises one or more isolating layers, for example only a single isolating layer. Preferentially, this/these layer(s) will be an isolating layer located between the core and the layer(s) of the shell comprising the active ingredient.

In another embodiment, the shell comprises one or more isolating layers, for example only a single isolating layer. Preferentially, this/these layer(s) will be a layer located after the layer of the shell comprising active layer furthest from the core.

In another embodiment, the shell comprises only two isolating layers. Preferentially, these layers will be an inner isolating layer in the shell which is located between the core and the layer(s) of the shell comprising the active ingredient and an outer isolating layer in the shell located after the layer comprising the active ingredient furthest from the core.

In another embodiment, the film-coated lozenge according to the invention intended to dissolve in the oral cavity comprises or consists of:
a core comprising at least one active ingredient and at least one cooked sugar, and
a shell comprising at least
   an isolating layer and
   a layer comprising at least one active ingredient.

In this embodiment, the at least one isolating layer is preferentially located between the core and said at least one layer comprising the active ingredient. The isolating layer is then adjacent to the core that is it is directly in contact with the core.

In one embodiment, the lozenge according to the invention comprises or consists of:
a core according to the invention,
a shell according to the invention consisting of successively:
   an inner isolating layer,
   a layer comprising at least one active ingredient, and
   an outer isolating layer.

In another embodiment, the lozenge according to the invention comprises or consists of:
a core according to the invention
a shell according to the invention consisting of sequentially:
   an isolating layer (adjacent to the core)
   a layer comprising at least one active ingredient, and
optionally an outer layer coating the shell.

In another embodiment, the lozenge according to the invention consists of:
a core according to the invention,
a shell according to the invention consisting of sequentially:
   a layer comprising at least one active ingredient,
   an isolating layer, and
optionally and outer layer coating the shell.

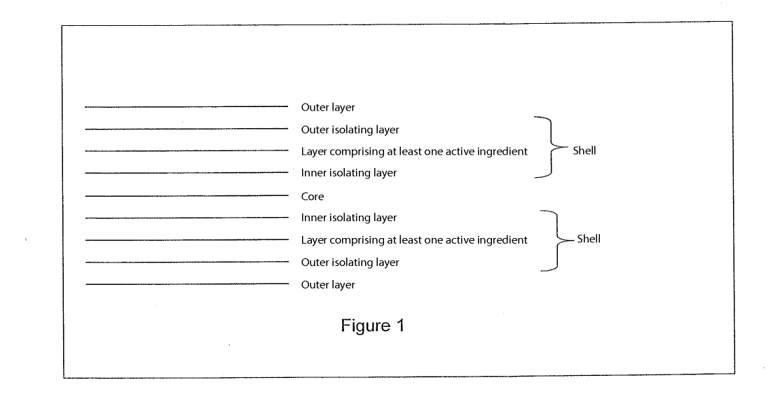

In another embodiment, illustrated in FIG. 1, the lozenge according to the invention consists of:
a core according to the invention,
a shell according to the invention consisting of successively:
   an inner isolating layer,
   a layer comprising at least one active ingredient,
   an outer isolating layer, and
an outer layer coating the shell.

All combinations, in number and arrangement, of the isolating layers and layers comprising at least one active ingredient are included in the present invention.

The core and the layer(s) of the shell comprising at least one active ingredient according to the invention can contain one, two, three or more active ingredients. The active ingredient(s) contained in the core and the layer(s) of the shell comprising at least one active ingredient can be identical or different. In one embodiment, the core and the layer(s) of the shell comprising at least one active ingredient each comprise a single active ingredient, preferentially nicotine as a salt. For example, the nicotine salt comprised in the core is nicotine resinate and the salt comprised in the layer of the shell comprising the active ingredient is nicotine bitartrate.

The inventors have found that it is highly advantageous to separate with isolating layers the layer comprising at least one active ingredient, from the core, and possibly from the outer layer coating the core and/or from the other layers comprising at least one active ingredient. Indeed, the presence of the isolating layers enables interactions to be avoided between the compounds/excipients of the different layers adjacent between them or with the core. For example, when the active ingredient contained in the layer(s) of the shell comprising at least one active ingredient is a nicotine salt, the isolating layers in the shell according to the invention have the advantage of isolating this nicotine salt from the buffering agent optionally contained in the core and/or in the outer layer coating the shell. Indeed, the inventors have observed that the contact between the nicotine salt and the buffering agent facilitates the desalting of the nicotine salt and formation of the base nicotine much more volatile than the salt. The presence of an isolating layer in the shell thus enables this transformation to be restricted and thus the evaporation of the nicotine from the rapidly dissolving film to be restricted. The presence of an isolating layer is further also particularly useful when the shell and/or the core of the lozenge comprise active ingredients which are different and in particular incompatible. For example, when the core comprises iron and the layer comprising the active ingredient comprises vitamin B9 (which is degraded in the presence of iron).

The presence of an isolating layer adjacent to the core has on the other hand the advantage of facilitating the production of the film-coated lozenge according to the invention by promoting adhesion of the following layers.

An isolating layer according to the invention preferentially comprises at least one film-forming polymer.

The inventors have besides discovered that it could be highly advantageous to select different nicotine salts for the core and the shell/film-coating of the lozenge according to the invention. In particular, the use of nicotine resinate in the core allows particularly a good heat resistance of the active ingredient. Nicotine ditartrate was in turn identified as being particularly suitable for the film-coating/shell because it allows a quick availability of the active and is besides particularly easy to bring into solution during the steps of manufacturing the lozenge.

Preferentially, the amount of base nicotine or base equivalent comprised in the layer of the shell comprising at least one active ingredient according to the invention will be between 9 and 50 weight % of the total amount of base nicotine or base equivalent comprised in the film-coated lozenge according to the invention, for example between 15 and 40%, further for example about 30%. For example, the core according to the invention will comprise between 1 and 5 mg pure nicotine, for example 1 mg, 2 mg, 2.5 mg or 4 mg and the layer of the shell comprising the active ingredient will comprise between 0.1 and 2 mg pure nicotine, for example 0.5 mg, 1 mg or 1.5 mg pure nicotine. In one embodiment, the total amount of nicotine present in the lozenge according to the invention will be comprised between 3 and 5 mg, for example 4 mg.

The film-coated lozenge according to the invention can further comprise an outer layer. By “outer layer” or “outer layer coating the shell” according to the invention, it is meant a layer coating the exterior of the shell according to the invention. This layer when present is thus directly in contact with the surrounding medium of the lozenge. The presence of an outer layer coating the shell is particularly considered when the film-coated lozenge according to the invention comprises one or more active ingredients which are sensitive in contact with air.

By “coating” according to the invention, it is meant that said shell fully covers the area of the core and the outer layer (if present) fully covers the area of the shell. By fully, it is understood according to the invention that the coverage is more than 95%, preferentially more than 99%, for example 100% of the area of the core or the shell respectively.

The layer(s) of the shell comprising at least one active ingredient and/or the isolating (outer or inner) layer(s) of the shell and/or the outer layer coating the shell preferentially comprise at least one, preferentially one, quickly disintegrating or film-forming polymer. In one embodiment, the polymer(s) present in the shell, in the layer(s) comprising at least one active ingredient, and the isolating (outer or inner) layers and/or the outer layer are identical.

The quickly disintegrating or film-forming polymer according to the invention is preferentially selected from the group comprising or consisting of copovidone, vinylpyrrolidone, vinyl acetate, hydroxypropylcellulose, hydroxypropylmethylcellulose, polyvinylpyrrolidone, macrogol, polyvinyl alcohol, methacrylic acid, ethyl acrylate copolymer (in a 1:1-1:2 ratio), hypromellose, shellac gum, hydroxyethylcellulose, amonio acrylate copolymer (type A and B), methacrylic acid-methyl methacrylate copolymer (in a 1:1-1:2 ratio), polyacrylate 30% dispersion, preferentially copovidone.

The outer layer coating the shell could further comprise one or more film-forming agents, plasticizers, opacifiers, anti-agglomerating agents, colorants, sweeteners, buffering agents, aromas. Preferentially, the outer layer does not comprise an active ingredient.

The amount of quickly disintegrating or film-forming polymer present in the layer of the shell comprising at least one active ingredient, the outer layer coating the shell and the isolating layer(s) in the shell will be preferentially between 55% and 75%, preferentially between 60 and 70%, for example around 65%, of the total solids contained in each of the different layers. These percentages enable in particular the isolating layers to ensure a good isolation between the layers with each other or with the core they separate and thus interactions between them to be avoided.

The outer layer coating the shell preferentially comprises a buffering agent, in particular when the active ingredient is nicotine. Preferentially, the buffering agent included in the outer layer will be such that it enables upon dissolving the outer layer, the pH of the oral cavity to be momentarily alkalinized between 8 and 10.5, for example between 9 and 10, in order to promote formation of the base nicotine from the nicotine salt contained in the lozenge according to the invention.

As a plasticizer according to the invention, there can be mentioned in particular macrogol, glycerol, triethyl citrate, dibutyl sebacate, triacetin, propylene glycol and triethyl acetate. In one embodiment, the plasticizer will be macrogol, glycerol, and/or propylene glycol. These plasticizers have in particular the advantage of not providing bitterness.

As an opacifier according to the invention, there can be named titanium dioxide.

As an anti-agglomerating agent according to the invention, there can be mentioned talc.

The colorant according to the invention can be any colorant in soluble or insoluble form in particular lake or iron oxide.

As a sweetener according to the invention, there can be named in particular sodium saccharin, acidic saccharin, aspartame, potassium acesulfame, sodium cyclamate, sucralose, neotame, alitame, stevia derivatives (for example stevia eupatoria or stevia rebaudiana). In one embodiment, the sweetener according to the invention will be potassium acesulfame or a mixture of potassium acesulfame and sodium saccharin.

As a buffering agent according to the invention, there can be named in particular calcium carbonate, sodium phosphate, sodium silicate, sodium citrate, sodium hydroxide, potassium hydroxide, sodium carbonate, sodium bicarbonate, potassium citrate, malic acid, disodium phosphate, ascorbic acid, citric acid, sodium lactate, tartaric acid, tetrapotassium phosphate, trisodium phosphate, preferentially sodium bicarbonate and sodium carbonate. Preferentially, the core according to the invention will comprise between 0 and 5% buffering agent, further preferentially between 0.5 and 2% buffering agent. Preferentially, the outer layer according to the invention will comprise between 5 and 20% buffering agent, preferentially between 8 and 15% buffering agent.

As an aroma, there can be in particular named all the aromas usable in lozenges, floral notes (including citrus fruit), blossom notes, licorice berry notes, anise, coffee chocolate, vanilla, caramel, mint as well as so-called “taste maskers” aromas. In one embodiment, the aroma will be selected from the aromas binding to the same gustatory receptors as nicotine such as mint aroma for example. When the active ingredient is nicotine, the presence of an aroma is preferred. Indeed surprisingly, the inventors have observed that the presence of an aroma in the outer layer coating the shell was sufficient to mostly mask the taste of nicotine released by the shell despite of the quick dissolution of the shell. In one embodiment, the core and the outer layer coating the shell comprise at least one aroma, preferentially the same aroma, for example mint. The aroma could for example be present between 95% to 98% in the core and between 2% to 5% in the outer layer coating the shell.

As an antioxidant agent according to the invention, there can be mentioned in particular ascorbic acid, citric acid monohydrate, fumaric acid, malic acid, araboascorbic acid, methionine, sodium ascorbate, sodium sulphite and metabisulphite, tartaric acid.

When the film-coated lozenge according to the invention comprises an antioxidant agent, the latter could be present in a proportion ranging from 0.01% to 1%.

In one embodiment, the lozenge according to the invention, in particular the lozenge according to the invention comprising nicotine as an active ingredient does not comprise an antioxidant agent.

In one embodiment, the dissolution time of the outer layer coating the shell and the shell is between 5 seconds and 5 minutes, preferentially lower than 3 minutes, further preferentially lower than 1 minute or 30 seconds.

The dissolution time corresponds to the release time of at least 70%, preferentially at least 90%, of the active ingredient(s) in the layers considered. The quick dissolution rate of the outer layer coating the shell and the shell can be measured by a dissolution test in a buffer at a physiological pH as in particular described in example 3.

In one embodiment, the presence of a shell and/or an outer layer coating the shell increases the release rate of nicotine with respect to the nicotine release rate from the core only, enabling nicotine to appear more quickly in the organism, without however changing Cmax or Tmax of nicotine. Tmax and Cmax can be readily determined by those skilled in the art by measuring nicotine amount in blood or plasma as a function of time after taking the lozenge according to the invention. It will be considered that Cmax of the nicotine kinetics is not changed by the presence of the shell and/or outer layer coating the shell with respect to the core only if Cmax values do not vary by more than 20%.

In a particular embodiment, the film-coated lozenge according to the invention comprises or consists of:
a core comprising or consisting of 0.1 to 10 dry weight % of nicotine resinate, 70 to 95 dry weight % of isomate, and advantageously 0.1 to 10 dry weight % of hypromellose, from 0 to 2 dry weight % of aspartame, from 0 to 2 dry weight % of potassium acesulfame, from 0 to 5 dry weight % of aromas, for example “Peppermint” and/or taste maskers from 0 to 2 dry weight % of sodium bicarbonate, from 0 to 4 dry weight % of anhydrous sodium carbonate, based on the total dry weight of the core;
a shell comprising or consisting of:
   a possible inner isolating layer comprising from 10 to 90 dry weight % of copovidone and advantageously from 10 to 90 dry weight % of talc (pharmaceutical grade), based on the total dry weight of said inner isolating layer;
   a layer comprising an active ingredient comprising or consisting of 0.1 to 10 dry weight % of nicotine ditartrate dihydrate, from 5 to 90 dry weight % of copovidone and from 10 to 90 dry weight % of talc (pharmaceutical grade), based on the total dry weight of said layer comprising an active ingredient;
   a possible outer isolating layer comprising or consisting of 10 to 90 dry weight % of copovidone and advantageously from 10 to 90 dry weight % of talc (pharmaceutical grade), based on the total dry weight of said inner isolating layer;
a possible outer layer coating the shell comprising or consisting of 10 to 90 dry weight % of copovidone and advantageously from 10 to 90 dry weight % of talc (pharmaceutical grade), from 0 to 30 dry weight % of titanium dioxide, from 0 to 30 dry weight % of potassium acesulfame, from 0 to 10 dry weight % of colorant, from 0 to 10 dry weight % of anhydrous sodium carbonate, from 0 to 20 dry weight % of sodium bicarbonate, from 0 to 20 dry weight % of aroma, based on the total dry weight of said outer layer.

Preferentially, the film-coated lozenge according to the present invention comprises or consists of:
a core comprising or consisting of 0.2 to 1 dry weight % of nicotine resinate, from 90 to 95 dry weight % of isomalt, and advantageously from 1 to 5 dry weight % of hypromellose, from 0.01 to 0.5 dry weight % of aspartame, from 0.01 to 0.5 dry weight % of potassium acesulfame, from 0.5 to 2 dry weight % of aromas, for example “Peppermint” and/or taste maskers, from 0.1 to 1 dry weight % of sodium bicarbonate, from 0.5 to 1 dry weight % of anhydrous sodium carbonate, based on the total dry weight of the core;
a shell comprising or consisting of:
   a possible inner isolating layer comprising or consisting of 50 to 80 dry weight % of copovidone and advantageously from 20 to 50 dry weight % of talc (pharmaceutical grade), based on the total dry weight of said inner isolating layer;
   a layer comprising an active ingredient comprising or consisting of 1 to 5 dry weight % of nicotine ditartrate dihydrate, from 40 to 80 dry weight % of copovidone and advantageously from 19 to 55 dry weight % of talc (pharmaceutical grade), based on the total dry weight of said layer comprising an active ingredient;
   a possible outer isolating layer comprising or consisting of 50 to 80 dry weight % of copovidone and advantageously from 20 to 50 dry weight % of talc (pharmaceutical grade), based on the total dry weight of said inner isolating layer;
a possible outer layer coating the shell comprising or consisting of 20 to 70 dry weight % of copovidone and advantageously from 10 to 50 dry weight % (pharmaceutical grade), from 5 to 15 dry weight % of titanium dioxide, from 1 to 10 dry weight % of potassium acesulfame, from 2 to 7 dry weight % of colorant, from 5 to 10 dry weight % of anhydrous sodium carbonate, from 1 to 10 dry weight % of sodium bicarbonate, from 0 to 10 dry weight % of aroma, based on the total dry weight of said outer layer.

More preferentially, the film-coated lozenge according to the present invention comprises or consists of:
a core comprising or consisting of 0.4 to 0.7 dry weight % of nicotine resinate, from 92 to 93 dry weight % of isomalt, and advantageously from 3 to 5 dry weight % of hypromellose, from 0.03 to 0.05 dry weight % of aspartame, from 0.05 to 0.07 dry weight % of potassium acesulfame, from 1.5 to 1.6 dry weight % of aromas, for example “Peppermint” and/or taste maskers, from 0.4 to 0.5 dry weight % of sodium bicarbonate, from 0.9 to 1 dry weight % of anhydrous sodium carbonate, based on the total dry weight of the core;
a shell comprising or consisting of:
   a possible inner isolating layer comprising or consisting of 60 to 70 dry weight % of copovidone and advantageously from 30 to 40 dry weight % of talc (pharmaceutical grade), based on the total dry weight % of said inner isolating layer;
   a layer comprising an active ingredient comprising or consisting of 3 to 5 dry weight % of nicotine ditartrate dihydrate, and advantageously from 70 to 80 dry weight % of copovidone and 20 to 30 dry weight % of talc (pharmaceutical grade), based on the total dry weight of said layer comprising an active ingredient;
   a possible outer isolating layer comprising or consisting of 60 to 75 dry weight % of copovidone and advantageously from 25 to 40 dry weight % of talc (pharmaceutical grade), based on the total dry weight of said inner isolating layer;
a possible outer layer comprising or consisting of 40 to 50 dry weight % of copovidone and advantageously from 10 to 20 dry weight % of talc (pharmaceutical grade), from 8 to 12 dry weight % of titanium dioxide, from 1.5 to 3.5 dry weight % of potassium acesulfame, from 4 to 6 dry weight % of colorant, from 6 to 8 dry weight % of anhydrous sodium carbonate, from 3 to 4.5 dry weight % of sodium bicarbonate, from 1 to 3 dry weight % of aroma, based on the total dry weight of said outer layer.

The present invention further relates to a method for preparing a film-coated lozenge according to the invention, comprising or consisting of the following steps:

a. preparing a core comprising at least one active ingredient and at least one cooked sugar according to the invention,

b. spraying and then drying a shell according to the invention on the core obtained in step a, and optionally,

c. spraying and then drying an outer layer according to the invention on the coated core obtained in step b.

In one embodiment, in the method according to the invention, step b can comprise or consist of the following successive steps:

b1. spraying and then drying at least one isolating layer according to the invention,

b2. spraying and then drying at least one layer comprising at least one active ingredient according to the invention,

b3. spraying and then drying at least one isolating layer according to the invention, preferentially an outer isolating layer.

Steps b1, b2 and/or b3 can be repeated as often as necessary to obtain the number of desired layers in the shell, for example from 1 to 5 times.

The present invention further relates to a film-coated lozenge according to the invention for use as a drug.

In one embodiment, the present invention relates to a lozenge according to the invention, in particular a lozenge comprising nicotine or one of the salts or complexes thereof for use in the treatment of tobacco addiction.

### EXAMPLES

### Example 1

### Obtaining a Lozenge comprising a Core, a Shell consisting of an Inner Isolating Layer, a Layer comprising Nicotine and an Outer Isolating Layer as well as an Outer Layer

### 1. Obtaining the Core of the Lozenge

### 2.1 Composition of the Core of the Lozenge A:

A core comprising the components described in table 1 is hot mixed with a sufficient amount of water for implementing the method. All the elements, including the matrix agent, water and sweetening base, except for the nicotine are mixed at a temperature of 90° C. (the temperature can range between 70 and 95° C., the boiling temperature). They are then all heated at 150° C. during the cooking. The nicotine is then added in the mass at a temperature of 120° C. (the temperature can range between 110 and 130° C.)

TABLE 1
composition of the core of the lozenge A

| | Amounts (mg) | |
|---|---|---|
| Components | (dry lozenges) | Dry percentage |
| 18% resinate nicotine | 13.89 (that is | 0.56% |
| | 2.5 mg pure | |
| | nicotine) | |
| Isomalt | 2310.36 | 92.41% |
| Hypromellose 6500 | 100 | 4% |
| Aspartame | 1 | 0.04% |
| Potassium acesulfame | 1.5 | 0.06% |
| Aromas and taste masker | 38.25 | 1.53% |
| Sodium bicarbonate | 11.67 | 0.47% |
| Anhydrous sodium carbonate | 23.33 | 0.93% |
| T.U.W | 2500 | 100% |

### 2. Preparation of the Solution to Form the Isolating Layer of the Shell

Copovidone (about 8%) is solubilized in purified water. About 3.7% talc is added to the solution and mixed by stirring. Stirring of the solution will be kept throughout the spraying step (mass percentage based on the mass of the isolating layer).

### 3. Spraying of the Inner Isolating Layer of the Shell

The cores of the shells obtained in step 1 are introduced in a Dumolin, Driacoater, Simo, Accela Cota type perforated film-coating turbine. The solution obtained in step 2 is sprayed onto the cores of the moving lozenges by adjusting the incoming air tempera denaturating the polyol contained in the core of the lozenge. This temperature can be between 55° C. and 65° C.

The thus film-coated lozenge is then dried at a temperature close to 40° C.

4. Preparation of the Solution to Form the Layer of the Shell comprising the Active Ingredient

Copovidone (about 8%) is solubilised in purified water. About 2.5% talc and 1.5365 g nicotine ditartrate are added to the solution and mixed by stirring. Stirring of the solution will be kept throughout the spraying step (mass percentage based on the mass of the layer comprising the active ingredient).

5. Spraying of the Layer of the Shell comprising the Active Ingredient

The cores of the lozenges film-coated with the isolating layer obtained in step 3 are introduced in a Dumoulin, Driacoater, Simo, Accela Cota type perforated film-coating turbine. The solution prepared in step 4 is sprayed onto these cores of moving film-coated lozenges by adjusting the incoming air temperature so as to extract the moisture generated and avoid denaturating the polyol contained in the core of the lozenge. This temperature can be between 55° C. and 65° C.

The thus film-coated lozenge is then dried at a temperature close to 40° C.

### 6. Preparation of the Solution to Form the Outer Isolating Layer of the Shell

Copovidone (about 8.5%) is solubilized in purified water. About 3.7% talc is added to the solution and mixed by stirring. Stirring of the solution will be kept throughout the spraying step (mass percentage based on the mass of the outer isolating layer of the shell).

### 7. Spraying of the Outer Isolating Layer of the Shell

The cores of film-coated lozenges obtained in step 5 are introduced in a Dumoulin, Driacoater, Simo, Accela Cota type perforated film-coating turbine. The solution prepared in step 4 is sprayed onto these cores of moving film-coated lozenges by adjusting the incoming air temperature so as to extract the moisture generated and avoid denaturating the polyol contained in the core of the lozenge. This temperature can be between 55° C. and 65° C.

The thus film-coated lozenge is then dried at a temperature close to 40° C.

### 8. Preparation of the Solution to Form the Outer Layer Coating the Shell

Copovidone (about 5.8%) is solubilized in purified water. 1.8% talc, 0.2% potassium acesulfame, 1.1% titanium dioxide, 0.6% colorant, 0.85% anhydrous sodium carbonate, 0.42% sodium bicarbonate and 0.22% aroma are successively added under stirring to the copovidone solution until complete solubilisation.

Stirring of the solution will be kept throughout the spraying step.

### 9. Spraying of the Outer Layer Coating the Shell

The cores of film-coated lozenges obtained in step 7 are introduced in a Dumoulin, Driacoater, Simo, Accela Cota type perforated film-coating turbine. The solution prepared in step 8 is sprayed onto these cores of moving film-coated lozenges by adjusting the incoming air temperature so as to extract the moisture generated and avoid denaturating the polyol contained in the core of the lozenge. This temperature can be between 55° C. and 65° C.

The thus film-coated lozenge is then dried at a temperature close to 40° C.

Lozenges containing several layers comprising at least one active ingredient and/or several isolating layers can be obtained by repeating the desired steps 2 to 7 after step 7 and as often as the desired number of quickly dissolving films.

### Example 2

### Lozenge obtained by the Method of the Example 2

The following film-coated lozenge A was obtained according to the method of example 1:

### 2.1. Composition of the Core of Lozenge A

The composition of the core is as described in table 1.

### 2.2. Composition of the Film Coating of Lozenge A

TABLE 2
composition of the film coating of lozenge A

| | | Amounts | Dry percentage |
|---|---|---|---|
| | Components | (mg) | for each layer |
| Inner isolating | COPOVIDONE | 26 | 68.42% |
| layer of the | TALC PHARMA M | 12 | 31.58% |
| shell | PURIFIED WATER | 290 | |
| Layer of the | DITARTRATE NICOTINE | 1.5 | 4.23% |
| shell | DIHYDRATE | | |
| comprising the | COPOVIDONE | 26 | 73.24% |
| active | TALC PHARMA M | 8 | 22.53% |
| ingredient | PURIFIED WATER | 290 | |
| Outer isolating | COPOVIDONE | 30 | 69.77% |
| layer of the | TALC PHARMA M | 13 | 30.23% |
| shell | PURIFIED WATER | 310 | |
| Outer layer | COPOVIDONE | 26 | 52.60% |
| coating the | TALC PHARMA M | 8 | 16.19% |
| shell | TITANUM DIOXIDE | 5 | 10.12% |
| | POTASSIUM | 1 | 2.02% |
| | ACESULFAME | 2.715 | 5.49% |
| | COLORANT | | |
| | ANHYDROUS SODIUM | 3.81 | 7.71% |
| | CARBONATE | | |
| | SODIUM BICARBONATE | 1.904 | 3.85% |
| | AROMA | 1 | 2.02% |
| | PURIFIED WATER | 400 | |

A film-coated lozenge B is obtained in the same way as lozenge A with the difference that 1 mg nicotine is present in the layer of the shell comprising the active ingredient.

As a comparative lozenge, a film-coated lozenge C is obtained in the same way as the film-coated lozenge B with the difference that the core of lozenge C does not contain an active ingredient (in order to obtain lozenges of the same weight (2 500 mg), the nicotine is replaced by the weight equivalent of polyol).

As a comparative lozenge, a lozenge D without a film coating is obtained, which only consists of the core of lozenge B.

### Example 3

### Release Kinetics of Nicotine

In order to measure the release time of nicotine by the film coating of the lozenge according to the invention, the release time of nicotine by lozenge C is compared to the release time of nicotine in lozenge D.

To do this, lozenges C and D are subjected to a dissolution test in a paddle apparatus (Eur. Ph.), in 500 ml of phosphate buffer at pH 7.0 (Eur. Ph) and at a temperature of 37° C. (conditions imitating the oral dissolution conditions). The lozenges are stirred at 100 rpm for 80 minutes. Dissolution medium is taken at 2.5, 5.0, 10, 15, 30, 60 and 80 minutes and the nicotine contained in this medium is assayed.

The nicotine is assayed by liquid chromatography at a flow rate of 1.0 ml/min (injected volume: 50 μl) (stationary phase: Xbridge Shield RP18 column, length 150 mm, diameter 4.6 mm, porosity 5.0 μm, temperature 50° C., mobile phase: 970 ml 20 mM KH2PO4 (adjusted at pH 6.5 with NH4OH)+30 ml acetonitrile) and UV detection at 254 nm.

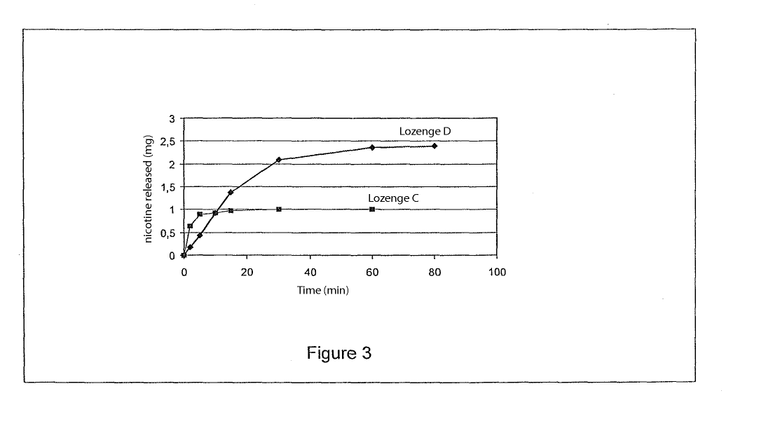

The experience was made 6 times. The average results obtained are represented in FIG. 3.

As can be observed, the nicotine release in lozenge C is very quick: it is initiated instantly, about 70% nicotine is released within 2.5 minutes and about 90% nicotine is released within 5 minutes.

On the contrary, the nicotine release by non-film-coated lozenge D begins more slowly (about 8% nicotine is released at 2.5 minutes and 16% at 5 minutes) and continues until about 60 minutes.

These results demonstrate the interest of lozenges according to the invention which combine quick nicotine release within the first minutes of dissolution and continuous nicotine release for more than 30 minutes.

1. A film-coated lozenge intended to dissolve in the oral cavity comprising:
a core comprising at least one active ingredient and at least one cooked sugar, and
a shell comprising at least
an isolating layer comprising at least one film-forming polymer and
a layer comprising at least one active ingredient.

2. The film-coated lozenge intended to dissolve in the oral cavity according to claim 1, characterized in that the at least one isolating layer is located between the core and said at least one layer comprising the active ingredient.

3. The film-coated lozenge according to claim 1 or 2, further comprising an outer layer coating the shell.

4. The film-coated lozenge according to any of claims 1 to 3, wherein the shell comprises at least two isolating layers.

5. The film-coated lozenge according to any of claims 1 to 4, wherein the shell successively consists of an inner isolating layer comprising at least one film-forming polymer, a layer comprising at least one active ingredient and an outer isolating layer comprising at least one film-forming polymer.

6. The film-coated lozenge according to any of claims 1 to 5, wherein the layer comprising at least one active ingredient and/or the outer layer coating the shell comprise a film-forming polymer.

7. The film-coated lozenge according to any of claims 1 to 6, wherein the film-forming polymer is selected from the group comprising copovidone, vinylpyrrolidone, vinyl acetate, hydroxypropylcellulose, hydroxypropylmethylcellulose, polyvinylpyrrolidone, macrogol, polyvinylalcohol, methacrylic acid, ethyl acrylate copolymer (in a 1:1-1:2 ratio), hypromellose, shellac gum, hydroxyethylcellulose, amonio methacrylate copolymer (type A and B), methacrylic acid-methyl methacrylate copolymer (in a 1:1-1:2 ratio) and polyacrylate 30% dispersion.

8. The film-coated lozenge according to any of claims 1 to 7, wherein the core and the layer of the shell comprising at least one active ingredient comprise a single active ingredient respectively.

9. The film-coated lozenge according to any of claims 1 to 8, wherein the active ingredient is nicotine as a salt, base or complex.

10. The film-coated lozenge according to claim 9, wherein the nicotine is selected from the group comprising nicotine bitartrate and nicotine resinate.

11. The film-coated lozenge according to any of claims 9 to 10, wherein the nicotine comprised in the core is nicotine resinate and the nicotine comprised in the layer of the shell comprising the active ingredient is nicotine bitartrate.

12. The film-coated lozenge according to any of claims 3 to 11, wherein the dissolution time of the outer layer coating the shell and of the shell is between 5 seconds and 5 minutes.

13. The film-coated lozenge according to any of claims 1 to 12, wherein the total dissolution time of the film-coated lozenge is between 15 and 30 minutes.

14. A method for preparing a film-coated lozenge according to any of claims 1 to 13, comprising the following steps:
a. preparing a core comprising at least one active ingredient and at least one cooked sugar,
b. spraying and then drying at least one shell on the core obtained in step a.
c. optionally, spraying and then drying an outer layer on the coated core obtained in step b.

15. The method for preparing a film-coated lozenge as claimed in claim 13, wherein step b comprises the following successive steps:
b1. spraying and then drying at least one isolating layer,
b2. spraying and then drying at least one layer comprising at least one active ingredient,
b3. spraying and then drying at least one isolating layer,
wherein steps b1 to b3 can be repeated as often as necessary.

16. The film-coated lozenge according to any of claims 1 to 13, for use as a drug.

17. The film-coated lozenge for use as claimed in claim 16, in the treatment of tobacco addiction.
